(19) United States
(12) Patent Application Publication
(10) Pub. No.: US 2017/0224789 A1
(43) Pub. Date: Aug. 10, 2017

(54) STABLE INJECTABLE COMPOSITION OF BIVALIRUDIN AND PROCESS FOR ITS PREPARATION

(71) Applicant: Piramal Enterprises Limited, Mumbai (IN)

(72) Inventors: Vandana SONAVARIA, Mumbai (IN); Shashwat BANERJEE, Thane (IN); Kamal Kumar UPADHYAY, Mumbai (IN)

(21) Appl. No.: 15/519,442

(22) PCT Filed: Oct. 15, 2015
(86) PCT No.: PCT/IB2015/057921

§ 371 Date: Apr. 14, 2017

(30) Foreign Application Priority Data
Oct. 16, 2014 (IN) 3296/MUM/2014

(51) Int. Cl. A61K 38/58; A61K 47/26; A61K 47/10; A61K 9/00

(52) U.S. Cl. CPC A61K 38/58; A61K 9/0019; A61K 47/26; A61K 47/10

(57) ABSTRACT

The present invention relates to a non-aqueous, stable and ready-to-use injectable composition of bivalirudin or pharmaceutically acceptable salt(s) or co-crystals thereof; and processes for its preparation. It is not required to reconstitute the injectable composition of bivalirudin with water prior to administration, thereby rendering it an easy-to-use injectable composition.

### FIELD OF THE INVENTION

The present invention relates to a stable, non-aqueous ready-to-use injectable composition of bivalirudin or a pharmaceutically acceptable salt or a co-crystal thereof; and processes for its preparation.

### BACKGROUND OF THE INVENTION

Anticoagulants (antithrombics, fibrinolytic, and thrombolytics) are a class of drugs that work to prevent the coagulation (clotting) of blood. They prevent deep vein thrombosis, pulmonary embolism, myocardial infarction and ischemic stroke. One class of anticoagulants is direct thrombin inhibitors (DTIs) that disrupt the activity of thrombin, an important protein in the coagulation cascade. Current members of this class include the bivalent drugs namely hirudin, lepirudin, and bivalirudin.

Bivalirudin, also known as Hirulog-8, is a synthetic congener of the naturally occurring thrombin peptide inhibitor hirudin, which is found in the saliva of the medicinal leech Hirudomedicinalis. Hirudin consists of 65 amino acids, although shorter peptide segments have proven to be effective as thrombin inhibitors. Bivalirudin is among these shorter peptides that demonstrate an anticoagulant activity. In contrast to hirudin, bivalirudin is a reversible inhibitor, which is ideal for temporary prevention of blood clotting during catherization procedures. It is frequently used for anticoagulation in the mainstream setting of invasive cardiology, particularly, percutaneous coronary intervention (PCI). It has a unique pharmacology profile; it undergoes predominant non-organ elimination (proteolysis), and has a short half-life of about 25 minutes.

Bivalirudin is a synthetic 20 amino acid residue peptide. Its chemical name is D-phenylalanyl-L-prolyl-L-arginyl-L-prolyl-glycyl-glycyl-glycyl-glycyl-L-asparagyl-glycyl-L-aspartyl-L-phenylalanyl-L-glutamyl-L- glutamyl-L-isoleucyl-L-prolyl-L-glutamyl-L-glutamyl-L-tyrosyl-L-leucine trifluoroacetate (salt) hydrate and has a molecular weight of 2180 daltons (as free base form) It has the structural formula as below:

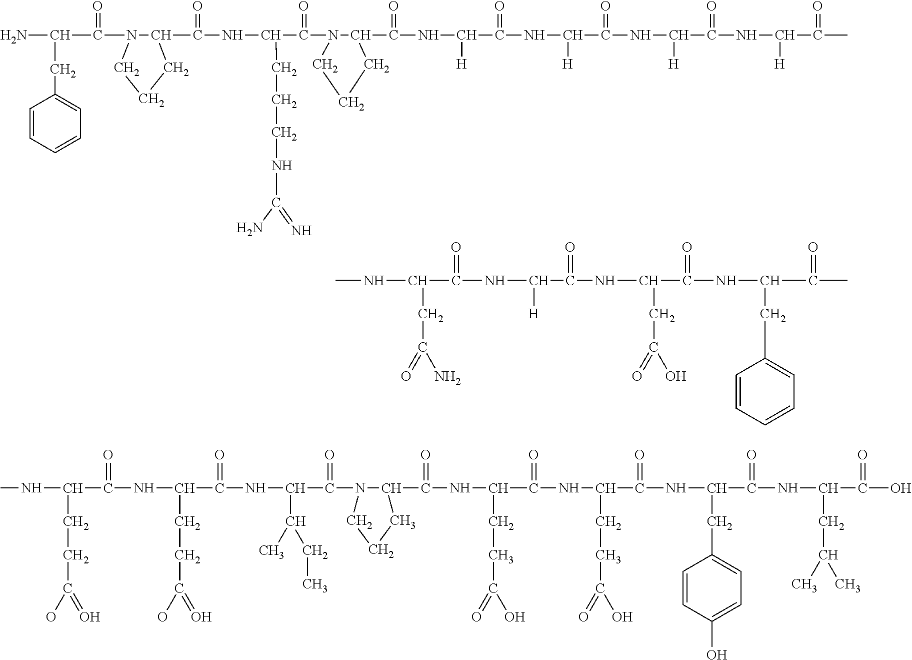

This medication is developed by The Medicine Co, having the approved indication treatment in patients with unstable angina undergoing percutaneous transluminal coronary angioplasty (“PTCA”); administration with the provisional use of glycol protein IIb/IIIa inhibitor for use as an anticoagulant in patients undergoing PCI; and treatment in patients with, or at risk of, heparin-induced thrombocytopenia (“HIT”) or heparin-induced thrombocytopenia and thrombosis syndrome (“HITTS”) undergoing PCI and marketed under the trade name ANGIOMAX® (bivalirudin for injection), in U.S. ANGIOMAX® is supplied in single-use vials as a white lyophilized cake containing 250 mg bivalirudin or 100 mg bivalirudin per vial.

This lyophilized product needs to be reconstituted with water for injection prior to administration and pH of the reconstituted injection is in the range of 5.0 to 6.0.

Bivalirudin was earlier disclosed in U.S. Pat. No. 5,196,404 as shorter peptides that demonstrate anticoagulant activity.

U.S. Pat. No. 7,582,727 and U.S. Pat. No. 7,598,343, discloses methods for preparing pharmaceutical compositions comprising bivalirudin and pharmaceutically acceptable carriers, wherein the composition involves reconstitution in an aqueous solution for injection.

U.S. Pat. No. 7,713,928 and U.S. Pat. No. 7,803,762, discloses ready-to-use bivalirudin composition comprising bivalirudin and one or more pharmaceutically acceptable stabilizing agents. The composition has a pH of about 4 to less than 5 and the total impurities are less than about 15% area-under-the-curve (“AUC”) after storage at 25° C. for 1 month. The concentration of bivalirudin as disclosed in the patents is between about 0.01 mg/mL and about 100 mg/mL. The examples disclose water as an essential ingredient of the composition.

U.S. Pat. No. 7,985,733 discloses a method for preventing the formation of a bivalirudin precipitate during the preparation of a pharmaceutical drug product comprising about 250 mg of bivalirudin. The method comprises: (i) preparing an aqueous solution comprising a buffer and a pH greater than the isoelectric point of bivalirudin; (ii) adding bivalirudin salt to the aqueous solution to form a bulk solution; (iii) transferring the bulk solution to one or more vessels; and (iv) drying the bulk solution. It is further stated that drying the bulk solution comprises lyophilizing.

Currently, bivalirudin is available in the form of a lyophilized composition that must be reconstituted prior to its administration. In general, reconstitution of the product involves multiple steps as the lyophilized cake is first reconstituted with water for injection, diluted and then administered.

Further, improper reconstitution may sometimes result in failure to provide the optimal dose to the patient. Furthermore, the lyophilized dosage form may incur high manufacturing cost and complexity of equipment. Moreover all the peptide based actives like bivalirudin are subject to degradation in aqueous solution. The specific type of degradation termed as deamidation at susceptible residues like glutamine (“Gln”) and asparagine (“Asn”), which are not stable in aqueous solution (Clinical Nutrition, Volume 10, Issue 4, August 1991, 186-192; Pharmaceutical ActaHelvetiae, 1999, Volume 74, Issue 1, 1-9). To overcome this problem, it is essential to find a solvent system in which bivalirudin has adequate solubility and stability.

Thus, there exists a need for the development of a new, improved composition that would prevent degradation yet increase solubility and stability of bivalirudin. Moreover, there is a need to provide a stable and ready-to-use injectable composition of bivalirudin to improve patient compliance.

As stated above use of aqueous solution in case of compositions of peptide based actives like bivalirudin has been a challenging task due to degradation of bivalirudin and impurity generation, which in turn makes the injection preparation unstable.

In consideration of the need as indicated above, the inventors of the present invention have done extensive research and conducted several experiments to develop a stable pharmaceutical non-aqueous, ready-to-use injectable composition of bivalirudin, without a need of reconstitution with water prior to administration, thereby rendering the composition according to the present invention an easy-to-use injectable composition. Further, being a non-aqueous injectable composition it is devoid of associated stability issues related to bivalirudin and involves a simple and cost effective process for preparation, which excludes the use of stabilizers such as preservative, anti-oxidants and polymers. The stable ready to use injectable composition in context of the instant invention is used for management of anti-coagulation in patients having unstable angina undergoing percutaneous transluminal coronary angioplasty (PTCA) patients undergoing percutaneous coronary intervention (PCI) and disease or condition associated with it.

### SUMMARY OF THE INVENTION

In one aspect, the present invention provides a non-aqueous, stable and ready-to-use injectable composition of bivalirudin or a pharmaceutically acceptable salt or a co-crystal thereof; wherein the said injectable composition comprises:
(i) bivalirudin or a pharmaceutically acceptable salt or a co-crystal thereof;
(ii) a non-aqueous solvent system;
(iii) optionally a polyol; and
(iv) a pH adjusting agent.

In one aspect, the present invention provides a non-aqueous, stable and ready-to-use injectable composition of bivalirudin or a pharmaceutically acceptable salt or a co-crystal thereof; wherein the said injectable composition comprises:
(i) bivalirudin or a pharmaceutically acceptable salt or a co-crystal thereof;
(ii) a non-aqueous solvent system consisting of a primary non-aqueous solvent and optionally one or more secondary non-aqueous co-solvent;
(iii) optionally a polyol; and
(iv) a pH adjusting agent.

In another aspect, the present invention provides a process for the preparation of a stable, non-aqueous and ready-to-use (RTU) injectable composition of bivalirudin or a pharmaceutically acceptable salt or a co-crystal thereof.

In a further aspect, the present invention provides a stable, non-aqueous, and ready-to-use injectable composition of bivalirudin or a pharmaceutically acceptable salt or a co-crystal thereof; for use as an anti-coagulant.

In another aspect, the present invention provides a stable, non-aqueous, and ready-to-use injectable composition of bivalirudin or a pharmaceutically acceptable salt or a co-crystal thereof; for the manufacture of a medicament for use as an anti-coagulant.

In another aspect, the present invention provides a stable, non-aqueous, and ready-to-use injectable composition of bivalirudin or a pharmaceutically acceptable salt or a co-crystal thereof; for use in the treatment of a subject having unstable angina undergoing percutaneous transluminal coronary angioplasty (PTCA), undergoing percutaneous coronary intervention (PCI) and disease or condition associated with it.

In an aspect of the present invention, the RTU bivalirudin composition comprises bivalirudin; a non-aqueous solvent system; optionally a polyol and a pH adjusting agent, wherein the composition is devoid of stabilizers such as preservatives, antioxidants and polymers.

In a still further aspect, the present invention provides a pharmaceutical kit comprising: (a) an injectable composition comprising of bivalirudin or a pharmaceutically acceptable salt or a co-crystal thereof; a non-aqueous solvent system comprising a primary non-aqueous solvent, optionally one or more secondary non-aqueous co-solvent; optionally a polyol; a pH adjusting agent; and (b) optionally a package insert comprising instructions for using the said injectable composition.

These and other aspects and advantages of the present invention will be apparent to those skilled in the art from the following description.

### DETAILED DESCRIPTION OF THE INVENTION

It should be understood that the detailed description and specific examples, while indicating embodiments of the invention, are given by way of illustration only, since various changes and modifications within the spirit and scope of the invention will become apparent to those skilled in the art. One skilled in the art, based upon the definitions herein, may utilize the present invention to its fullest extent. The following specific embodiments are to be construed as merely illustrative, and not limitative of the remainder of the disclosure in any way whatsoever.

Unless otherwise defined, all the terms used herein, including the technical and scientific terms, have the meaning as that generally understood by one of ordinary skill in the art to which the present invention relates.

### Definitions

For the purpose of the disclosure, listed below are definitions of various terms used to describe the present invention. Unless otherwise indicated, these definitions apply to the terms as they are used throughout the specification and the appended claims, either individually or as part of a larger group. They should not be interpreted in the literal sense. They are not general definitions and are relevant only for this application.

It should be noted that, as used in this specification and the appended claims, the singular forms “a”, “an” and “the” include plural referents unless the content clearly dictates otherwise.

It should be noted that the term “or” is generally employed in its sense including “and/or” unless the content clearly dictates otherwise.

As used herein, the term “about” means approximately and, in the context of numerical values, the term “about” can be construed to estimate a value that is ±10% of the value or range recited.

Within the context of the present invention the term “stable” as used herein in reference to the injectable composition of bivalirudin or a pharmaceutically acceptable salt or a co-crystal thereof; means that the said composition does not exhibit degradation upon storage over a set time limit, at a set temperature, and at an identified pH; or within the context of the present invention the term “stable” as used herein in reference to the injectable composition of bivalirudin or a pharmaceutically acceptable salt or a co-crystal thereof; means that the said composition exhibit a chromatographic purity, wherein the impurities identified are within the acceptable limit (i.e. not more than 7%).

Within the context of the present invention, the term “sterile composition” means one in which essentially all forms of microbial life have been destroyed by an appreciable amount to meet the sterilization criteria outlined in the US Pharmacopeia.

Within the context of the present invention, the term “ready-to-use” or “RTU” as used herein in reference to the injectable composition of bivalirudin or a pharmaceutically acceptable salt or a co-crystal thereof; is a sterile, non-aqueous, injectable composition that is stable and not reconstituted from a lyophilizate. The term “RTU” also encompasses within its scope, sterile, non-aqueous, injectable composition that is stable and has been diluted from a concentrated, liquid solution just prior to use.

Within the context of the present invention the term “non-aqueous composition” as used herein means a composition with not more than 2% water content.

The term “non-aqueous solvent” means a non-polar solvent which contain bonds between atoms of similar electronegativity like carbon and hydrogen by which they lack partial charges and do not contain hydrogen attached to oxygen or nitrogen so that they are unable to form hydrogen bonds with themselves. Examples of solvents are selected from the group but not limited to polyethylene glycols (PEGs), propylene glycol (PG), dipropylene glycol, tripropylene glycol, ethylene glycol, polyvinylpyrrolidone (PVP), methoxy propylene glycol (MPEG), glycerol and glycofurol or a mixture thereof.

The term “non-aqueous RTU composition” means the composition is devoid of any water content in the final finished product or during process for preparation of the same. However, a negligible amount i.e. not more than 2% of water or moisture may be present due to external environmental factors, which does not have any impact on the physiochemical property, specifically on the stability of the composition.

As used herein, the term “has not been reconstituted from a lyophilizate” means that a solid has not been dissolved or suspended.

The term “pharmaceutically acceptable excipient” as used herein means a diluent, carrier, or composition auxiliary, which is non-toxic, and inert, and which does not have undesirable effects on a subject to whom it is administered and is suitable for delivering a therapeutically active agent (e.g. bivalirudin) to the target site without affecting the therapeutic activity of the said active agent.

The term “pharmaceutically acceptable salt” or “pharmaceutically acceptable salt(s)” means salt(s) of bivalirudin, which can be prepared by treating bivalirudin with an appropriate acid or a base. Examples of pharmaceutically acceptable base addition salts include, but are not limited to, sodium, potassium, calcium, magnesium, ammonium salts or an organic base salt. Examples of pharmaceutically acceptable organic base addition salts include, but are not limited to, those derived from organic bases such as lysine, arginine, guanidine, and the like. Examples of pharmaceutically acceptable acid addition salts include, but are not limited to, those derived from inorganic acids such as hydrochloric acid, nitric acid, phosphoric acid, sulfuric acid and the like, as well as the salts derived from organic acids such as acetic acid, trifluoroacetic acid, propionic acid, oxalic acid, maleic acid, benzoic acid, succinic acid, fumaric acid, phthalic acid, benzenesulfonic acid, p-toluenesulfonicacid, citric acid, tartaric acid, methanesulfonic acid and the like.

The term “co-crystal” refers to a crystalline structure made up of two or more components in a definite stoichiometric ratio, where each component is defined as either an atom, ion, or molecule. The term co-crystal” encompasses within its scope many types of compounds, including hydrates, solvates and clathrates.

The term “composition” or “injectable composition” refers to a unit dose or a multi dose of an active pharmaceutical ingredient and a pharmaceutically acceptable excipient, which can be prepared by the processes described in one or more embodiments of the present invention. In the context of the present invention, the terms “composition”, “injectable compositions” and “non-aqueous, stable and ready-to-use injectable composition” are used interchangeably. In the injectable composition of the present invention, the active pharmaceutical ingredient is bivalirudin or a pharmaceutically acceptable salt or a co-crystal thereof.

The term “polyol” as used herein, refers to an alcohol containing multiple hydroxyl groups. Polyols may comprise, but are not limited to, glycerin, sucrose, lactose, glucose, fructose, arabinose, xylose, ribose, mannose, galactose, dextrose, sorbose, sorbitol, mannitol, maltose, cellobiose, xylitol, or a combination thereof.

The term “stirring” encompasses within its scope, sonication or turbulence or agitation by other means. Therefore the term “stirring” can be interchangeably used with the terms “sonication”, “turbulence” or “agitation”.

As used herein, the term “pH” is a measure of hydrogen ion concentration, as commonly used in the art. Customarily, the pH provides a measure on a scale from 0 to 14 of the acidity or alkalinity of a solution. In the context of the present invention, the pH of the injectable composition of bivalirudin or a pharmaceutically acceptable salt or a co-crystal thereof, of the present invention is between about 5.0 and about 8.0.

The term “pH adjusting agent” or “pH adjusting agents” as used herein, includes a substance that adjusts the pH of pharmaceutical compositions to intended pH. Customarily, the pH adjusting agents may include pharmaceutically acceptable acids, bases, or buffering agents. For example, the acids may include, but are not limited to, citric acid, fumaric acid, gluconic acid, lactic acid, malic acid, metatartaric acid, tartaric acid, benzene sulphonic acid, ascorbic acid, citric acid and the like. In the context of the present invention, the pH adjusting agent may be a base or a buffering agent. The bases may be one or more inorganic bases or organic bases, including, but not limited to, alkaline carbonate, alkaline bicarbonate, alkaline earth metal carbonate, alkaline hydroxide, alkaline earth metal hydroxide or amine. For example, the inorganic or organic base may be an alkaline hydroxide such as lithium hydroxide, potassium hydroxide, cesium hydroxide, sodium hydroxide or the like; an alkaline carbonate such as calcium carbonate, sodium carbonate or the like; or an alkaline bicarbonate such as sodium bicarbonate or the like; the organic base may also be sodium acetate. The buffering agent can be, but is not limited to an alkali metal salt of an amino acid, aluminum hydroxide, aluminum magnesium hydroxide, aluminum glycinate, calcium acetate, calcium bicarbonate, calcium borate, calcium carbonate, calcium citrate, calcium gluconate, calcium glycerophosphate, calcium hydroxide, calcium lactate, calcium phthalate, calcium phosphate, calcium succinate, calcium tartarate, dibasic sodium phosphate, dipotassium hydrogen phosphate, dipotassium phosphate, disodium hydrogen phosphate, disodium succinate, magnesium acetate, magnesium aluminate, magnesium borate, magnesium bicarbonate, magnesium carbonate, magnesium citrate, magnesium gluconate, magnesium hydroxide, magnesium lactate, magnesium metasilicate aluminate, magnesium oxide, magnesium phthalate, magnesium phosphate, magnesium silicate, magnesium succinate, magnesium tartarate, potassium acetate, potassium carbonate, potassium bicarbonate, potassium borate, potassium citrate, potassium metaphosphate, potassium phthalate, potassium phosphate, potassium polyphosphate, potassium pyrophosphate, potassium succinate, potassium tartarate, sodium acetate, sodium bicarbonate, sodium borate, sodium carbonate, sodium citrate, sodium gluconate, sodium hydrogen phosphate, sodium lactate, sodium phthalate, sodium phosphate, sodium polyphosphate, sodium pyrophosphate, sodium sesquicarbonate, sodium succinate, sodium tartarate, sodium tripolyphosphate, tetrapotassium pyrophosphate, tetrasodium pyrophosphate, tripotassium phosphate, trisodium phosphate, or a mixture thereof. A relative pH has been measured because it is difficult to measure the absolute pH of a non-aqueous solution due to lack of hydrogen ion activity or concentration. Further, the pH of the composition may vary depending upon the type of instrument and dilution media.

In the context of the invention the term “solvent system” refers to a primary solvent and optionally one or more secondary solvent selected from a group of solvents.

As used herein, the term “absolute alcohol” refers to ethanol containing from about 98.0-99.8 v/v/% of ethanol and from about 0.2-0.5 v/v % of water.

Within the context of the present invention and as used herein the term “subject” refers to an animal, preferably a mammal, and most preferably a human. In the context of the present invention, the term “mammal” is used interchangeably with the term “patient” or “subject”. In the context of the present invention the phrase “a subject in need thereof” means a subject (patient) in need for the treatment of a disease or disorder for which an anticoagulant can be suitably used.

### Injectable Composition:

As discussed herein above, the inventors of the present invention have done extensive research and conducted several experiments to develop a stable pharmaceutical injectable composition of bivalirudin or a pharmaceutically acceptable salt or a co-crystal thereof; which can be prepared in a solubilized and stable form suitable for ready-to-use injection.

Further, as a RTU composition, the composition of the present invention has enhanced patient compliance and also provides a more stable, safe and effective composition when compared to its counterpart, the currently marketed lyophilized ANGIOMAX® composition. Furthermore, being a non-aqueous composition, it has added advantages in comparison to aqueous RTU injection composition in terms of bivalirudin stability which is unstable in aqueous media and requires stabilizers such as preservatives, anti-oxidants and polymers, in turn make the aqueous composition complicated and expensive.

In respect of the injectable composition of bivalirudin or a pharmaceutically acceptable salt or a co-crystal thereof; of the present invention, there is no requirement of reconstituting the composition with water prior to its administration, thus eliminating tedious task of reconstitution step in aseptic area, thereby providing an easy-to-use injectable composition.

Accordingly, in one aspect, the present invention relates to a stable, non-aqueous, and ready-to-use injectable composition of bivalirudin or a pharmaceutically acceptable salt or a co-crystal thereof; wherein the said injectable composition comprises:
(i) bivalirudin or a pharmaceutically acceptable salt or a co-crystal thereof;
(ii) a non-aqueous solvent system consisting of a primary non-aqueous solvent, and optionally one or more secondary non-aqueous co-solvent;
(iii) optionally a polyol and
(iv) a pH adjusting agent.

In an embodiment, the injectable composition contains bivalirudin or a pharmaceutically acceptable salt or a co-crystal thereof; at a concentration in the range from about 1 mg/mL to about 250 mg/mL.

In an embodiment, the injectable composition contains bivalirudin or a pharmaceutically acceptable salt or a co-crystal thereof; at a concentration in the range from about 10 mg/mL to about 100 mg/mL.

In an embodiment, the injectable composition contains bivalirudin or a pharmaceutically acceptable salt or a co-crystal thereof; at a concentration in the range from about 25 mg/mL to about 60 mg/mL.

In an embodiment, the injectable composition contains bivalirudin or a pharmaceutically acceptable salt or a co-crystal thereof at a concentration of about 50 mg/mL.

In an embodiment, the non-aqueous solvent system comprises 100% primary non-aqueous solvent; or in the non-aqueous solvent system, the primary non-aqueous solvent and the secondary non-aqueous co-solvent can be used in a ratio ranging from about 99:1 to about 50:50.

In an embodiment, the non-aqueous solvent system comprises 100% primary non-aqueous solvent.

In an embodiment, in the non-aqueous solvent system, the primary non-aqueous solvent and the secondary non-aqueous co-solvent can be used in the ratio ranging from about 99:1 to about 50:50.

In an embodiment, in the non-aqueous solvent system, the primary non-aqueous solvent and the secondary non-aqueous co-solvent can be used in the ratio of 99.1, 95:5, 90:10, 85:15, 80:20, 70:30, 60:40 or 50:50.

In an embodiment, in the non-aqueous solvent system, the primary non-aqueous solvent and the secondary non-aqueous co-solvent can be used in the ratio of 90:10.

In an embodiment, in the non-aqueous solvent system, the primary non-aqueous solvent and the secondary non-aqueous co-solvent can be used in the ratio of 85:15.

In an embodiment the non-aqueous solvent system comprises one or more solvent selected from the group consisting of propylene glycol, glycerol, polyethylene glycol, methanol, ethanol, absolute alcohol, 1-propanol and isopropanol (isopropyl alcohol) or a mixture thereof.

In an embodiment, the primary non-aqueous solvent contained in the non-aqueous solvent system is selected from the group consisting of but not limited to propylene glycol, glycerol and polyethylene glycol or a mixture thereof.

In an embodiment, the primary non-aqueous solvent is propylene glycol.

In an embodiment, the secondary optional non-aqueous co-solvent contained in the non-aqueous solvent system is a (C₁-C₃)alkyl containing alcohol selected from the group consisting of but not limited to methanol, ethanol, absolute alcohol, 1-propanol and isopropanol (isopropyl alcohol) or a mixture thereof.

In an embodiment, the secondary optional non-aqueous co-solvent contained in the non-aqueous solvent system is isopropyl alcohol, ethanol or absolute alcohol; or a combination thereof.

In an embodiment, the secondary optional non-aqueous co-solvent contained in the non-aqueous solvent system is ethanol

In another embodiment, the secondary optional non-aqueous co-solvent contained in the non-aqueous solvent system is absolute alcohol.

In another embodiment, the secondary optional non-aqueous co-solvent contained in the non-aqueous solvent system is isopropyl alcohol.

In another embodiment, the secondary optional non-aqueous co-solvent contained in the non-aqueous solvent system is a combination of ethanol/absolute alcohol and isopropyl alcohol.

In an embodiment, the polyol is selected from a group consisting of but not limited to glycerin, sucrose, lactose, glucose, fructose, arabinose, xylose, ribose, mannose, galactose, dextrose, sorbose, sorbitol, mannitol, maltose, cellobiose, xylitol, trehalose or a combination thereof. In an embodiment, the polyol is in the range of about 0.01% to about 10% of the total injectable composition of bivalirudin or a pharmaceutically acceptable salt or a co-crystal thereof.

In an embodiment, the polyol is sorbitol or racemic salts or isomers thereof.

In an embodiment, the polyol is D-sorbitol.

In the non-aqueous solvent system used in the injectable composition of bivalirudin or a pharmaceutically acceptable salt or a co-crystal thereof; the primary non-aqueous solvent, the co-solvent and the polyol are present in an amount such that bivalirudin at the concentration of at least 25 mg/ml bivalirudin is completely soluble and stable in the injectable composition.

In an embodiment, the non-aqueous solvent system contains propylene glycol, ethanol, sorbitol and isopropyl alcohol.

In an embodiment, the non-aqueous solvent system contains propylene glycol, ethanol and sorbitol.

In an embodiment, the non-aqueous solvent system contains propylene glycol, absolute alcohol, sorbitol and isopropyl alcohol.

In an embodiment, the non-aqueous solvent system contains propylene glycol, absolute alcohol and sorbitol.

In an embodiment, the non-aqueous solvent system comprises 100% propylene glycol; or in the non-aqueous solvent system, the propylene glycol and the ethanol/absolute alcohol (and/or isopropyl alcohol) can be used in a ratio ranging from about 99:1 to about 50:50.

In an embodiment, the non-aqueous solvent system comprises 100% propylene glycol.

In an embodiment, in the non-aqueous solvent system, propylene glycol and ethanol/absolute alcohol (and/or isopropyl alcohol) can be used in the ratio ranging from about 99:1 to about 50:50.

In an embodiment, in the non-aqueous solvent system, propylene glycol and ethanol/absolute alcohol (and/or isopropyl alcohol) can be used in the ratio of 99:1, 95:5, 90:10, 85:15, 80:20, 70:30, 60:40 or 50:50.

In an embodiment, in the non-aqueous solvent system, propylene glycol and ethanol/absolute alcohol (and/or isopropyl alcohol) can be used in the ratio of 90:10.

In an embodiment, in the non-aqueous solvent system, propylene glycol and ethanol/absolute alcohol (and/or isopropyl alcohol) can be used in the ratio of 85:15.

In an embodiment, in the non-aqueous solvent system, comprises 100% propylene glycol.

In another embodiment, the pH adjusting agent is selected from pharmaceutically acceptable acids, bases, or buffering agents.

In an embodiment, the pH adjusting agent is sodium hydroxide.

In another embodiment, the pH of the ready-to-use bivalirudin injectable composition of the present invention is between about 5.0 and about 8.0.

In an embodiment, the RTU bivalirudin composition comprises bivalirudin; a non-aqueous solvent system; optionally a polyol and a pH adjusting agent, wherein the composition is devoid of stabilizers such as preservatives, antioxidants and polymers.

### Process for the Preparation of Injectable Composition:

In one aspect, the present invention relates to a process for the preparation of a non-aqueous, stable and ready-to-use injectable composition of bivalirudin or a pharmaceutically acceptable salt or a co-crystal thereof, comprising the steps of:
a) dissolving a pH adjusting agent in a non-aqueous solvent system consisting of a primary non-aqueous solvent to obtain a first solution;
b) optionally adding polyol to the first solution of step (a) under constant stirring until the polyol dissolves to obtain a second solution;
c) optionally adding secondary non-aqueous solvent to the second solution of step (b) to obtain a third solution;
d) adding bivalirudin to the third solution of step (c) to obtain a clear solution;
e) filtering the clear solution of step (d) to obtain a sterile clear solution; and
g) filling the sterile clear solution of step (e) into a container to obtain a composition in a ready-to-use form.

In an embodiment, the present invention relates to a process for the preparation of the non-aqueous, stable and ready-to-use injectable composition of bivalirudin or a pharmaceutically acceptable salt or a co-crystal thereof; wherein the said process comprises the steps of:
a) dissolving a pH adjusting agent in a non-aqueous solvent system consisting of a primary non-aqueous solvent to obtain a mixture and stirring the resulting mixture at a temperature ranging from 2° C. to 60° C. over a period of 30 minutes to 120 minutes to obtain a first solution;
b) allowing the resulting first solution of step (a) to attain a temperature of 2° C. to room temperature;
c) optionally adding polyol to the first solution of step (b) under constant stirring until the polyol dissolves, to obtain a second solution;
d) optionally adding a secondary non-aqueous solvent to the second solution of step (c) under constant stirring for 5 minutes to 10 minutes to obtain a third solution;
e) adding bivalirudin to the third solution of step (d) clear solution;
f) filtering the clear solution as obtained in step (e) one or more times to obtain a clear sterile solution; and
g) filling the clear solution of step (f) in suitable containers to obtain a composition in a ready-to-use form.
   In another aspect, the present invention relates to a process for the preparation of a non-aqueous, stable and ready-to-use injectable composition of bivalirudin or a pharmaceutically acceptable salt or a co-crystal thereof, comprising the steps of:
a) dissolving polyol and pH adjusting agent in primary non-aqueous solvent to obtain a first solution;
b) optionally adding a secondary non-aqueous solvent to the first solution of step (a) to obtain a second solution;
c) adding bivalirudin to the second solution of step (b) to obtain a clear solution;
d) filtering the clear solution of step (c) one or more times to obtain a sterile clear solution; and
e) filling the clear solution of step (d) into a container to obtain a composition in a ready-to-use form.

In an embodiment, the present invention relates to a process for the preparation of the non-aqueous, stable and ready-to-use injectable composition of bivalirudin or a pharmaceutically acceptable salt or a co-crystal thereof; wherein the said process comprises the steps of:
a) dissolving polyol and pH adjusting agent in primary non-aqueous solvent to obtain a solution by stirring the resulting mixture at a temperature ranging from 2° C. to 60° C. over a period of 30 minutes to 120 minutes to obtain a first solution;
b) allowing the resulting first solution of step (a) to attain a temperature of 2° C. to room temperature;
c) optionally adding a secondary non-aqueous solvent to the first solution of step (b) under constant stirring for 5 minutes to 10 minutes to obtain a second solution;
d) adding bivalirudin to the second solution of step (c) to obtain a clear solution.
e) filtering the clear solution of step (d) one or more times to obtain a sterile clear solution; and
f) filling the sterile clear solution of step (e) in suitable containers to obtain a composition in a ready-to-use form.

In an embodiment, in the process for the preparation of the injectable composition of bivalirudin or a pharmaceutically acceptable salt or a co-crystal thereof; the non-aqueous solvent system consists of a primary non-aqueous solvent and a secondary non-aqueous co-solvent.

In an embodiment, in the process for the preparation of the injectable composition of bivalirudin or a pharmaceutically acceptable salt or a co-crystal thereof; the non-aqueous solvent system comprises 100% primary non-aqueous solvent; or in the non-aqueous solvent system, the primary non-aqueous solvent and the secondary non-aqueous co-solvent can be used in a ratio ranging from about 99:1 to about 50:50.

In an embodiment, in the process for the preparation of the injectable composition of bivalirudin or a pharmaceutically acceptable salt or a co-crystal thereof; the non-aqueous solvent system comprises 100% primary non-aqueous solvent.

In an embodiment, in the process for the preparation of the injectable composition of bivalirudin or a pharmaceutically acceptable salt or a co-crystal thereof; in the non-aqueous solvent system, the primary non-aqueous solvent and the secondary non-aqueous co-solvent can be used in the ratio ranging from about 99:1 to about 50:50.

In an embodiment, in the process for the preparation of the injectable composition of bivalirudin or a pharmaceutically acceptable salt or a co-crystal thereof; the non-aqueous solvent system comprises 100% primary non-aqueous solvent or the primary non-aqueous solvent and the secondary non-aqueous co-solvent can be used in the ratio of 99:1, 95:5, 90:10, 85:15, 80:20, 70:30, 60:40 or 50:50.

In an embodiment, in the process for the preparation of the injectable composition of bivalirudin or a pharmaceutically acceptable salt or a co-crystal thereof; the non-aqueous solvent system comprises 100% primary non-aqueous solvent or the primary non-aqueous solvent and the secondary non-aqueous co-solvent can be used in the ratio of 90:10.

In an embodiment, in the process for the preparation of the injectable composition of bivalirudin or a pharmaceutically acceptable salt or a co-crystal thereof; the non-aqueous solvent system comprises 100% primary non-aqueous solvent or the primary non-aqueous solvent and the secondary non-aqueous co-solvent can be used in the ratio of 85:15.

In an embodiment, in the process for the preparation of the injectable composition of bivalirudin or a pharmaceutically acceptable salt or a co-crystal thereof; the primary non-aqueous solvent contained in the non-aqueous solvent system is selected from the group consisting of propylene glycol, glycerol and polyethylene glycol or a mixture thereof.

In an embodiment, in the process for the preparation of the injectable composition of bivalirudin or a pharmaceutically acceptable salt or a co-crystal thereof; the primary non-aqueous solvent is propylene glycol.

In an embodiment, in the process for the preparation of the injectable composition of bivalirudin or a pharmaceutically acceptable salt or a co-crystal thereof; the secondary non-aqueous co-solvent is a (C₁-C₃)alkyl containing alcohol selected from the group consisting of methanol, ethanol, absolute alcohol, 1-propanol and isopropanol (isopropyl alcohol) or a mixture thereof.

In an embodiment, in the process for the preparation of the injectable composition of bivalirudin or a pharmaceutically acceptable salt or a co-crystal thereof; the secondary non-aqueous co-solvent is ethanol or absolute alcohol.

In an embodiment, in the process for the preparation of the injectable composition of bivalirudin or a pharmaceutically acceptable salt or a co-crystal thereof; the secondary non-aqueous co-solvent is ethanol.

In another embodiment, in the process for the preparation of the injectable composition of bivalirudin or a pharmaceutically acceptable salt or a co-crystal thereof; the secondary non-aqueous co-solvent is absolute alcohol.

In an embodiment, in the process for the preparation of the injectable composition of bivalirudin or a pharmaceutically acceptable salt or a co-crystal thereof; the polyol is selected from a group consisting of glycerin, sucrose, lactose, glucose, fructose, arabinose, xylose, ribose, mannose, galactose, dextrose, sorbose, sorbitol, mannitol, maltose, cellobiose, xylitol, trehalose or a combination thereof.

In an embodiment, in the process for the preparation of the injectable composition of bivalirudin or a pharmaceutically acceptable salt or a co-crystal thereof; the polyol is in the range of about 0.01% to about 10% of the total injectable composition of bivalirudin.

In an embodiment, in the process for the preparation of the injectable composition of bivalirudin or a pharmaceutically acceptable salt or a co-crystal thereof; the polyol is sorbitol or racemic salts or isomers thereof.

In an embodiment, in the process for the preparation of the injectable composition of bivalirudin or a pharmaceutically acceptable salt or a co-crystal thereof; the polyol is D-sorbitol.

In an embodiment, in the process for the preparation of the injectable composition of a peptide drug or a pharmaceutically acceptable salt or a co-crystal thereof; the non-aqueous solvent system comprises 100% propylene glycol.

In an embodiment, in the process for the preparation of the injectable composition of bivalirudin or a pharmaceutically acceptable salt or a co-crystal thereof; the non-aqueous solvent system comprises propylene glycol and ethanol.

In an embodiment, in the process for the preparation of the injectable composition of bivalirudin or a pharmaceutically acceptable salt or a co-crystal thereof; the non-aqueous solvent system comprises propylene glycol and absolute alcohol.

In an embodiment, in the process for the preparation of the injectable composition of a peptide drug or a pharmaceutically acceptable salt or a co-crystal thereof; the non-aqueous solvent system comprises propylene glycol and isopropyl alcohol.

In an embodiment, in the process for the preparation of the injectable composition of a peptide drug or a pharmaceutically acceptable salt or a co-crystal thereof; the non-aqueous solvent system comprises 100% propylene glycol; or in the non-aqueous solvent system, the propylene glycol and ethanol/absolute alcohol (and/or isopropyl alcohol) can be used in a ratio ranging from about 99:1 to about 50:50.

In an embodiment, in the process for the preparation of the injectable composition of a peptide drug or a pharmaceutically acceptable salt or a co-crystal thereof; the non-aqueous solvent system comprises 100% propylene glycol.

In an embodiment, in the process for the preparation of the injectable composition of bivalirudin or a pharmaceutically acceptable salt or a co-crystal thereof; in the non-aqueous solvent system, the propylene glycol and ethanol is in the ratio ranging from about 99:1 to about 50:50.

In an embodiment, in the process for the preparation of the injectable composition of bivalirudin or a pharmaceutically acceptable salt or a co-crystal thereof; in the non-aqueous solvent system, propylene glycol and ethanol can be used in the ratio of 99:1, 95:5, 90:10, 85:15, 80:20, 70:30, 60:40 or 50:50.

In an embodiment, in the process for the preparation of the injectable composition of bivalirudin or a pharmaceutically acceptable salt or a co-crystal thereof; in the non-aqueous solvent system, propylene glycol and ethanol can be used in the ratio of 90:10.

In an embodiment, in the process for the preparation of the injectable composition of bivalirudin or a pharmaceutically acceptable salt or a co-crystal thereof; in the non-aqueous solvent system, propylene glycol and ethanol can be used in the ratio of 85:15.

In an embodiment, in the process for the preparation of the injectable composition of bivalirudin or a pharmaceutically acceptable salt or a co-crystal thereof;

the pH adjusting agent is selected from pharmaceutically acceptable acids, bases, or buffering agents.

In an embodiment, in the process for the preparation of the injectable composition of bivalirudin or a pharmaceutically acceptable salt or a co-crystal thereof; the pH adjusting agent is sodium hydroxide or potassium hydroxide.

In an embodiment, in the process for the preparation of the injectable composition of bivalirudin or a pharmaceutically acceptable salt or a co-crystal thereof; the pH adjusting agent sodium hydroxide is present in a concentration range from about 4.00 mg/mL to about 6.5 mg/mL to optimize the pH of bivalirudin injection 50 mg/mL.

In another embodiment, the pH of the ready-to-use bivalirudin injectable composition obtained by the process as described above is between about 5.0 and about 8.0.

### Method of Use of the Injectable Composition:

In an aspect, the present invention relates to use of the non-aqueous, stable and ready-to-use injectable composition of bivalirudin or a pharmaceutically acceptable salt or a co-crystal thereof as an anticoagulant, wherein the said injectable composition is as described herein above in one or more embodiments of the present invention.

In another aspect, the present invention relates to use of the non-aqueous, stable and ready-to-use injectable composition of bivalirudin or a pharmaceutically acceptable salt or a co-crystal thereof; for the manufacture of a medicament as an anticoagulant; wherein the said injectable composition is as described herein above in one or more embodiments of the present invention.

In an embodiment, the present invention relates to use of the non-aqueous, stable and ready-to-use injectable composition of bivalirudin or a pharmaceutically acceptable salt or a co-crystal thereof; for the manufacture of a medicament for the treatment in patients undergoing percutaneous coronary intervention (PCI); wherein the said injectable composition is as described herein above in one or more embodiments of the present invention.

In an embodiment, the present invention relates to use of the non-aqueous, stable and ready-to-use injectable composition of bivalirudin or a pharmaceutically acceptable salt or a co-crystal thereof; wherein the PCI is percutaneous transluminal coronary angioplasty.

In another embodiment, the present invention relates to use of the non-aqueous, stable and ready-to-use injectable composition of bivalirudin or a pharmaceutically acceptable salt or a co-crystal thereof; for the manufacture of a medicament for treating a subject with unstable angina undergoing percutaneous transluminal coronary angioplasty.

In another embodiment, the present invention relates to use of the non-aqueous, stable and ready-to-use injectable composition of bivalirudin or a pharmaceutically acceptable salt or a co-crystal thereof; for the manufacture of a medicament as a glycoprotein IIb/IIIa inhibitor for treating a subject undergoing percutaneous coronary intervention.

In another embodiment, the present invention relates to a method of treatment comprising administering to a subject in need thereof a therapeutically effective amount of the non-aqueous, stable and ready-to-use injectable composition of bivalirudin or a pharmaceutically acceptable salt or a co-crystal thereof; wherein the said injectable composition is as described in one or more embodiments of the present invention as described herein above.

In another embodiment, the non-aqueous, stable and ready-to-use injectable composition of bivalirudin or a pharmaceutically acceptable salt or a co-crystal thereof; can be packaged in a suitable container depending upon the composition and the method of administration of the composition. Suitable containers known to a person skilled in the art include vials, ampoules and infusion bag.

In another embodiment, the present invention provides a pharmaceutical kit comprising the non-aqueous, stable and ready-to-use injectable composition of bivalirudin or a pharmaceutically acceptable salt or a co-crystal thereof; wherein the said composition comprises of bivalirudin or a pharmaceutically acceptable salt or a co-crystal thereof; a non-aqueous solvent system consisting of a primary non-aqueous solvent, a secondary non-aqueous co-solvent and a polyol; and a pH adjusting agent. The kit may further comprise a package insert, including information about the indication, usage, doses, direction for administration, contraindications, precautions and warnings. The kit may further contain optional materials for storing and/or administering the composition for example, an infusion bag as well as instructions for its storage and use.

In another embodiment, the non-aqueous, stable and ready-to-use injectable composition of bivalirudin or a pharmaceutically acceptable salt or a co-crystal thereof; can be delivered to the subject intravenously. Methods of delivering the RTU injectable compositions intravenously are well known in the art.

In another embodiment, the non-aqueous, stable and ready-to-use injectable composition of bivalirudin or a pharmaceutically acceptable salt or a co-crystal thereof; can be delivered to the subject by infusion. For example, the injectable dosage form can be delivered intravenously through infusion.

It is understood that modifications that do not substantially affect the activity of the various embodiments of this invention are included within scope of the invention disclosed herein. It will be evident to one skilled in the art that the present invention is not limited to the above description or illustrative examples provided below, and that it can be embodied in other specific forms without departing from the essential attributes thereof. It is therefore desired that the description and examples be considered in all respects as illustrative and not restrictive, reference being made to the appended claims, and all changes which come within the meaning and range of equivalency of the claims are therefore intended to be embraced therein. Accordingly, the following examples are intended to illustrate but not to limit the scope of the present invention.

### EXAMPLES

### Example 1

The injectable composition of bivalirudin (RTU), 50 mg/mL

| Ingredients | mg/mL |
|---|---|
| Bivalirudin | 50 |
| Propylene Glycol | 988 |
| Ethanol | 39.45 |
| D-Sorbitol | 0.1093 |
| Sodium Hydroxide | qs* |

*qs: quantity sufficient

### Procedure:

a) Sodium hydroxide was dissolved in propylene glycol to obtain a solution, by stirring the resulting mixture at a temperature ranging from room temperature to 65° C. over a period of 30 minutes to 120 minutes,
b) The resulting solution obtained from step (a) was allowed to attain a temperature of 15-35° C.
c) Sorbitol was added to step (b) solution under constant stirring till it was dissolved.
d) Ethanol was added to the solution of step (c) under constant stirring for 5 minutes to 10 minutes to obtain a solution.
e) Bivalirudin was added and allowed to solubilize in the solution of step (d).
f) The dispersion as obtained in step (e) was subjected to turbulence for 30-120 min and filtered one or more times through a filter to obtain a clear solution,
g) The clear solution of step (f) was filled in suitable containers to obtain a composition in a ready-to-use form.

### Example 2

The injectable composition of bivalirudin (RTU), 50 mg/mL

| Ingredients | mg/mL |
|---|---|
| Bivalirudin | 50 |
| Propylene Glycol | 988 |
| Ethanol | 39.45 |
| D-Sorbitol | 0.1093 |
| Sodium Hydroxide | qs* |

*qs: quantity sufficient

### Procedure:

a) Sorbitol and sodium hydroxide were dissolved in propylene glycol to obtain a solution, by stirring the resulting mixture at a temperature ranging from room temperature to 65° C. over a period of 30 minutes to 120 minutes.
b) The resulting solution obtained from step (a) was allowed to attain a temperature of 20° C. to 25° C.
c) Ethanol was added to the solution of step (b) under constant stirring for 5 minutes to 10 minutes to obtain a solution.
d) Bivalirudin was added and allowed to disperse in the solution of step (c).
e) The dispersion as obtained in step (d) was subject to turbulence for 30-120 minutes and filtered one or more times through filter to obtain a clear solution,
f) The clear solution of step (e) was filled in suitable containers to obtain a composition in a ready-to-use form.

The compositions described in the following examples (3 to 11) are prepared by following the same procedure as described in the above example 1.

### Example 3

The injectable composition of bivalirudin (RTU), 50 mg/mL

| Ingredients | mg/mL |
|---|---|
| Bivalirudin | 50 |
| Propylene Glycol | 936 |
| Ethanol | 78.90 |
| D-Sorbitol | 0.2186 |
| Sodium Hydroxide | qs* |

*qs: quantity sufficient

### Example 4

The injectable composition of bivalirudin (RTU), 50 mg/mL

| Ingredients | mg/mL |
|---|---|
| Bivalirudin | 50 |
| Propylene Glycol | 884 |
| Ethanol | 118.35 |
| D-Sorbitol | 0.3279 |
| Sodium Hydroxide | qs* |

*qs: quantity sufficient

### Example 5

The injectable composition of bivalirudin (RTU), 50 mg/mL

| Ingredients | mg/mL |
|---|---|
| Bivalirudin | 50 |
| Propylene Glycol | 832 |
| Ethanol | 157.80 |
| D-Sorbitol | 0.4372 |
| Sodium Hydroxide | qs* |

*qs: quantity sufficient

### Example 6

The injectable composition of bivalirudin (RTU), 30 mg/mL

| Ingredients | mg/mL |
|---|---|
| Bivalirudin | 30 |
| Propylene Glycol | 988 |
| Ethanol | 39.45 |
| Sorbitol | 0.1093 |
| Sodium Hydroxide | qs* |

*qs: quantity sufficient

### Example 7

The injectable composition of bivalirudin (RTU), 60 mg/mL

| Ingredients | mg/mL |
|---|---|
| Bivalirudin | 60 |
| Propylene Glycol | 988 |
| Ethanol | 39.45 |
| Sorbitol | 0.1093 |
| Sodium Hydroxide | qs* |

*qs: quantity sufficient

### Example 8

The injectable composition of bivalirudin (RTU), 50 mg/mL

| Ingredients | mg/mL |
|---|---|
| Bivalirudin | 50 |
| Propylene Glycol | 728 |
| Ethanol | 236.7 |
| D-Sorbitol | 0.6558 |
| Sodium Hydroxide | qs* |

*qs: quantity sufficient

### Example 9

The injectable composition of bivalirudin (RTU), 50 mg/mL

| Ingredients | mg/mL |
|---|---|
| Bivalirudin | 50 |
| Propylene Glycol | 624 |
| Ethanol | 315.6 |
| D-Sorbitol | 0.8744 |
| Sodium Hydroxide | qs* |

*qs: quantity sufficient

### Example 10

The injectable composition of bivalirudin (RTU), 50 mg/mL

| Ingredients | mg/mL |
|---|---|
| Bivalirudin | 50 |
| Propylene Glycol | 416 |
| Ethanol | 473.4 |
| D-Sorbitol | 1.3116 |
| Sodium Hydroxide | qs* |

*qs: quantity sufficient

### Example 11

The injectable composition of bivalirudin (RTU), 50 mg/mL

| Ingredients | mg/mL |
|---|---|
| Bivalirudin | 50 |
| Propylene Glycol | 416 |
| Iso propyl alcohol | 315.6 |
| Ethanol | 157.8 |
| D-Sorbitol | 1.3116 |
| Sodium Hydroxide | qs* |

*qs: quantity sufficient

### Example 12

The injectable composition of bivalirudin (RTU), 50 mg/mL

| Ingredients | mg/mL |
|---|---|
| Bivalirudin | 50 |
| Propylene Glycol | 1040 |
| Sodium Hydroxide | qs* |

*qs: quantity sufficient

### Procedure:

a) Sodium Hydroxide was dissolved in propylene glycol to obtain a solution, at room temperature to 65° C. over a period of 30 minutes to 120 minutes,
b) The resulting solution obtained from step (a) was allowed to attain a temperature of 20-25° C.
c) Bivalirudin was added and allowed to disperse on the solution of step (b).
d) The dispersion as obtained in step (c) was homogenized for 15 minutes at 4200 rpm and stirred for 120 minutes and filtered one or more times through PES filter to obtain a clear solution,
e) The clear solution of step (d) was filled in suitable containers to obtain a composition in a ready-to-use form.

### Example 12

### Stability Studies:

The bivalirudin injection 50 mg/mL lab batch are packed in Clear, USP type 1, 5 mL siliconized and nonsiliconised glass vial with 20 mm Teflon and FluroTec coated rubber closure sealed with flip off and placed for evaluating stability in both upright and inverted position. Stability studies are being performed at 5° C.±3° C. per the ICH guideline. The results of the ongoing stability studies of lab scale batch are compared with the initial results of RLD, ANGIOMAX®. The lyophilized ANGIOMAX® is reconstituted with 5 mL sterile water for injection to achieve final concentration of 50 mg/mL prior to analysis. An additional stability study is also performed at 15° C. The stability at 15° C. is conducted to address the effect of short term excursions outside the proposed label storage condition, e.g., during shipping or handling. The shelf life of the bivalirudin RTU injection (50 mg/mL) has been studied before admixture based on the real time data (room temperature 5° C.±3° C.) and bivalirudin (5 mg/mL & 0.5 mg/mL) after admixture with 0.9% sodium chloride for injection and 5.0% Dextrose in water for 24 h and 48 h.

### A.: Before Admixture

Before admixture storage stability assessment was performed on RTU bivalirudin injection of Example 1 (solvent system comprising primary and secondary solvent) by means of HPLC.

### Experimental Conditions:

Mobile Phase A: 6.8 g of potassium dihydrogen phosphate in 1000 mL of water

### Mobile Phase B: Acetonitrile

| Column | Zorbax XDB C18 (150 × 4.6 mm), 5 μm | |
|---|---|---|
| Column temp | 25° | C. |
| Flow rate | 1.0 | mL/min |
| Wavelength | 215 | nm |
| Injection volume | 10 | μL |
| Run time | 10 | minute. |
| Sample temp. | 10° | C. |

### Gradient Program:

| Time | % Mobile phase A | % Mobile Phase B |
|---|---|---|
| 0 | 100 | 0 |
| 1 | 100 | 0 |
| 2 | 40 | 60 |
| 4 | 40 | 60 |
| 6 | 100 | 0 |
| 10 | 100 | 0 |

Comparative impurity profiling of known impurities of RLD was done with proposed bivalirudin injection 50 mg/ml filled in 5 ml vials with FluroTec or Teflon coated closure system. The stability results of the critical parameter such as related substance and assay of the lab scale development batches are summarized below:

TABLE NO. 1
Stability results for the injectable composition of bivalirudin (RTU),

| Test | | | | | | | | | |
|---|---|---|---|---|---|---|---|---|---|
| | | FLUROTEC | | | | TEFLON | | | |
| | Reference | | 2M/ | 3M/ | 6M/ | | 2M/ | 3M/ | 6M/ |
| RRT | (RLD) | Initial | 2-8 C. | 2-8 C. | 2-8 C. | Initial | 2-8 C. | 2-8 C. | 2-8 C. |

Description

| | | | | | | | | |
|---|---|---|---|---|---|---|---|---|
| Clear | Clear | Clear | Clear | Clear | Clear | Clear | Clear | Clear |
| solution | solution | solution | solution | solution | solution | solution | solution | solution |

Chromatographic purity
Known impurities

| | | | | | | | | | | |
|---|---|---|---|---|---|---|---|---|---|---|
| BIVA | 0.40 | 0.07 | 0.04 | 0.08 | 0.06 | 0.05 | 0.06 | 0.08 | 0.08 | 0.04 |
| (12-20) | | | | | | | | | | |
| PLUS- | 0.96 | 0.14 | 0.03 | 0.03 | 0.03 | 0.00 | 0.11 | 0.07 | 0.00 | 0.00 |
| GLY | | | | | | | | | | |
| DES- | 1.07 | 0.02 | 0.04 | 0.04 | 0.03 | 0.04 | 0.14 | 0.03 | 0.08 | 0.01 |
| GLY | | | | | | | | | | |
| BETA- | 1.14 | 0.03 | 0.00 | 0.00 | 0.00 | 0.00 | 0.00 | 0.00 | 0.00 | 0.00 |
| ASP9 | | | | | | | | | | |
| ALPHA- | 1.19 | 0.23 | 0.06 | 0.05 | 0.04 | 0.05 | 0.00 | 0.08 | 0.08 | 0.03 |
| ASP9 | | | | | | | | | | |

Unknown impurities

| | | | | | | | | | | |
|---|---|---|---|---|---|---|---|---|---|---|
| UNK | 0.08 | ND | ND | ND | ND | ND | ND | ND | ND | ND |
| UNK | 0.16 | 0.03 | 0.04 | 0.13 | 0.10 | 0.16 | 0.02 | 0.07 | 0.11 | 0.13 |
| UNK | 0.18 | ND | 0.02 | ND | 0.05 | 0.06 | ND | 0.03 | 0.04 | 0.04 |
| UNK | 0.27 | ND | ND | ND | ND | ND | ND | ND | ND | ND |
| UNK | 0.44 | 0.10 | 0.19 | 0.46 | 0.39 | 0.53 | 0.09 | 0.27 | 0.44 | 0.45 |
| UNK | 0.51 | ND | ND | ND | ND | ND | ND | ND | ND | ND |
| UNK | 0.54 | 0.01 | ND | ND | ND | ND | ND | ND | ND | 0.03 |
| UNK | 0.57 | 0.03 | ND | ND | ND | ND | ND | ND | ND | ND |
| UNK | 0.61 | 0.02 | ND | ND | ND | ND | ND | ND | ND | 0.06 |
| UNK | 0.63 | ND | ND | ND | ND | ND | ND | ND | ND | ND |
| UNK | 0.65 | ND | ND | ND | ND | ND | ND | ND | ND | ND |
| UNK | 0.68 | ND | ND | ND | ND | ND | ND | ND | ND | ND |
| UNK | 0.7 | ND | ND | ND | ND | ND | ND | ND | ND | ND |
| UNK | 0.75 | ND | ND | ND | ND | ND | ND | 0.02 | ND | ND |
| UNK | 0.81 | ND | ND | 0.03 | ND | ND | ND | 0.03 | 0.04 | ND |
| UNK | 0.85 | ND | 0.05 | ND | ND | ND | ND | ND | ND | ND |
| UNK | 0.92 | 0.15 | 0.05 | 0.16 | 0.04 | 0.10 | ND | 0.08 | 0.11 | 0.03 |
| UNK | 0.97 | 0.11 | ND | ND | ND | ND | ND | ND | ND | ND |
| UNK | 1.04 | 0.12 | ND | ND | ND | ND | ND | ND | ND | ND |
| UNK | 1.13 | 0.01 | ND | ND | ND | ND | ND | ND | ND | ND |
| UNK | 1.15 | 0.03 | ND | ND | ND | ND | ND | ND | ND | ND |
| UNK | 1.29 | 0.09 | 0.15 | 0.31 | 0.25 | 0.36 | 0.15 | 0.21 | 0.29 | 0.36 |
| UNK | 1.3 | ND | ND | ND | ND | ND | ND | ND | ND | ND |
| UNK | 1.33 | ND | ND | ND | ND | ND | ND | ND | ND | 0.01 |
| UNK | 1.36 | ND | ND | ND | ND | ND | ND | ND | 0.08 | ND |
| UNK | 1.47 | ND | ND | 0.07 | ND | ND | ND | ND | 0.09 | ND |
| UNK | 1.51 | ND | ND | ND | ND | ND | ND | ND | ND | ND |
| UNK | 1.54 | ND | 0.03 | ND | 0.05 | 0.08 | ND | ND | ND | 0.07 |
| UNK | 1.57 | ND | ND | ND | ND | ND | ND | ND | ND | ND |
| UNK | 1.6 | ND | ND | ND | ND | 0.16 | ND | ND | ND | ND |
| UNK | 1.65 | ND | ND | ND | 0.12 | 0.24 | ND | ND | 0.10 | 0.19 |
| Total Imp | — | 1.20 | 0.72 | 1.36 | 1.16 | 1.83 | 0.57 | 0.96 | 1.54 | 1.46 |

ND: Not Detected,
UNK: Unknown &
RRT: Relative Retention Time

### B. After Admixture

The dilution stability study for 24 h and 48 h at 25° C. for 5 mg/mL and 0.5 mg/mL of bivalirudin injection was performed with 0.9% Sodium chloride for injection and 5% Dextrose in water. Example 1 (solvent system comprising primary and secondary solvent) by means of HPLC.

TABLE 2
Stability data of Bivalirudin RTU (5 mg/mL & 0.5 mg/mL) injection and
RLD (5 mg/mL) after dilution with 0.9% Sodium chloride for injection

| | | 0.9% NaCI for injection | | | | | |
|---|---|---|---|---|---|---|---|
| Name of | | 5 MG/ML | 5 MG/ML | 5 MG/ML | 0.5 MG/ML | 0.5 MG/ML | 0.5 MG/ML |
| Impurity | RRT | INI | 24 HRS | 48 HRS | INI | 24 HRS | 48 HRS |

Chromatographic purity
Known Impurities

| | | | | | | | |
|---|---|---|---|---|---|---|---|
| BIVA (12-20) | 0.40 | 0.12 | 0.09 | 0.09 | 0.12 | 0.13 | 0.08 |
| PLUS-GLY | 0.96 | 0.03 | ND | ND | ND | ND | ND |
| DES-GLY | 1.07 | 0.04 | ND | 0.03 | ND | ND | ND |
| BETA-ASP9 | 1.14 | 0.00 | ND | ND | ND | ND | ND |
| ALPHA-ASP9 | 1.19 | 0.12 | 0.14 | 0.16 | ND | 0.11 | ND |

Un Known Impurities

| | | | | | | | |
|---|---|---|---|---|---|---|---|
| UNK | 0.16 | 0.05 | 0.06 | 0.05 | ND | 0.04 | 0.05 |
| UNK | 0.18 | ND | 0.04 | 0.01 | ND | 0.03 | 0.11 |
| UNK | 0.44 | 0.14 | 0.18 | 0.22 | 0.23 | 0.19 | 0.22 |
| UNK | 0.54 | ND | ND | ND | ND | ND | ND |
| UNK | 0.57 | 0.05 | ND | ND | ND | ND | ND |
| UNK | 0.61 | 0.03 | 0.03 | ND | ND | ND | ND |
| UNK | 0.81 | ND | ND | ND | ND | ND | ND |
| UNK | 0.92 | 0.05 | 0.06 | 0.05 | ND | ND | ND |
| UNK | 1.29 | 0.11 | 0.19 | 0.30 | ND | 0.17 | 0.33 |
| UNK | 1.36 | ND | ND | ND | ND | ND | ND |
| UNK | 1.54 | ND | ND | ND | ND | ND | ND |
| UNK | 1.65 | ND | ND | ND | ND | ND | ND |
| Total Imp | | 0.73 | 0.78 | 0.93 | 0.34 | 0.67 | 0.78 |

ND: Not Detected,
UNK: Unknown &
RRT: Relative Retention Time

TABLE 3
Stability data of bivalirudin RTU (5 mg/mL & 0.5 mg/mL) injection
and RLD (5 mg/mL) after dilution with 5.0% Dextrose in Water

| | | 5% Dextrose in water | | | | | |
|---|---|---|---|---|---|---|---|
| Name of | | 5 MG/ML | 5 MG/ML | 5 MG/ML | 0.5 MG/ML | 0.5 MG/ML | 0.5 MG/ML |
| Impurity | RRT | INI | 24 HRS | 48 HRS | INI | 24 HRS | 48 HRS |
| BIVA (12-20) | 0.40 | 0.07 | 0.06 | 0.08 | 0.06 | 0.18 | 0.09 |
| PLUS-GLY | 0.96 | ND | ND | ND | ND | ND | ND |
| DES-GLY | 1.07 | ND | ND | ND | ND | ND | ND |
| BETA-ASP9 | 1.14 | ND | ND | ND | ND | ND | ND |
| ALPHA-ASP9 | 1.19 | 0.11 | 0.10 | 0.12 | ND | 0.29 | 0.10 |
| UNK | 0.16 | 0.03 | 0.06 | 0.07 | 0.06 | 0.07 | 0.07 |
| UNK | 0.18 | ND | ND | ND | ND | ND | ND |
| UNK | 0.44 | 0.11 | 0.18 | 0.27 | 0.13 | 0.19 | 0.23 |
| UNK | 0.54 | 0.03 | ND | ND | ND | ND | ND |
| UNK | 0.57 | 0.04 | 0.03 | ND | ND | ND | ND |
| UNK | 0.61 | ND | 0.03 | ND | ND | ND | ND |
| UNK | 0.81 | ND | ND | ND | ND | ND | ND |
| UNK | 0.92 | 0.05 | 0.04 | ND | ND | ND | ND |
| UNK | 1.29 | 0.10 | 0.16 | 0.31 | ND | 0.15 | 0.17 |
| UNK | 1.36 | ND | ND | ND | ND | ND | ND |
| UNK | 1.54 | 0.06 | ND | ND | ND | ND | ND |
| UNK | 1.65 | ND | ND | ND | ND | ND | ND |
| Total Imp | | 0.60 | 0.66 | 0.84 | 0.25 | 0.87 | 0.66 |

ND: Not Detected
UNK: Unknown

### Example 13

Storage stability assessment was performed on RTU Bivalirudin injection of Example 1 (solvent system comprising single solvent) by means of HPLC.

TABLE 4

| | | | |
|---|---|---|---|
| Test | RRT | INITIAL | 2-8° C. 2M |

Chromatographic purity
Known Impurity

| | | | |
|---|---|---|---|
| BIVA (12-20) | 0.40 | 0.06 | 0.05 |
| PLUS-GLY | 0.96 | 0.03 | 0.00 |
| DES-GLY | 1.07 | 0.03 | 0.00 |
| BETA-ASP9 | 1.14 | 0.00 | 0.00 |
| ALPHA-ASP9 | 1.19 | 0.04 | 0.04 |

Un known impurity

| | | | |
|---|---|---|---|
| UNK | 0.16 | 0.00 | 0.04 |
| UNK | 0.19 | 0.01 | 0.00 |
| UNK | 0.44 | 0.02 | 0.17 |
| UNK | 0.54 | 0.00 | 0.00 |
| UNK | 0.59 | 0.01 | 0.00 |
| UNK | 0.62 | 0.01 | 0.00 |
| UNK | 0.92 | 0.04 | 0.00 |
| UNK | 1.29 | 0.02 | 0.11 |
| UNK | 1.54 | 0.00 | 0.00 |
| UNK | 1.65 | 0.00 | 0.10 |
| UNK | 1.69 | 0.03 | 0.00 |
| Total Imp | | 0.31 | 0.51 |

### CONCLUSION

It was observed that the five established known impurities listed in related substance specification and unknown impurities are found to be within acceptable limit i.e. not more than 7% in the proposed composition. Hence, it can be concluded that the non-aqueous and ready-to-use injectable composition of bivalirudin of the instant invention is stable.

1. A stable, non-aqueous and ready-to-use injectable composition of bivalirudin or a pharmaceutically acceptable salt or a co-crystal thereof; comprising:
(i) bivalirudin or a pharmaceutically acceptable salt or a co-crystal thereof;
(ii) a non-aqueous solvent system;
(iii) optionally a polyol; and
(iv) a pH adjusting agent.

2. The injectable composition according to claim 1, wherein the non-aqueous solvent system comprises a primary non-aqueous solvent.

3. The injectable composition according to claim 1, wherein the non-aqueous solvent system comprises of a primary non-aqueous solvent and one or more secondary non-aqueous co-solvents.

4. The injectable composition according to claim 1, wherein the non-aqueous solvent system comprises one or more solvent selected from the group consisting of propylene glycol, dipropylene glycol, tripropylene glycol, ethylene glycol, glycerol, polyethylene glycol, methanol, ethanol, absolute alcohol, 1-propanol and isopropanol (isopropyl alcohol) or a mixture thereof.

5. The injectable composition according to claim 2, wherein the primary non-aqueous solvent system comprises a solvent selected from the group consisting of propylene glycol, glycerol and polyethylene glycol or a mixture thereof.

6. The injectable composition according to claim 3, wherein the secondary non-aqueous solvent system comprises solvent(s) selected from a group consisting of methanol, ethanol, absolute alcohol, 1-propanol and isopropanol (isopropyl alcohol) or a mixture thereof.

7. The injectable composition according to claim 1, wherein the polyol is selected from the group consisting of glycerin, sucrose, lactose, glucose, fructose, arabinose, xylose, ribose, mannose, galactose, dextrose, sorbose, sorbitol, mannitol, maltose, cellobiose, xylitol, trehalose or a combination thereof.

8. The injectable composition according to claim 1, wherein the pH adjusting agent is used in a quantity sufficient to adjust the final pH of the composition, which ranges from about 4.00 mg/mL to about 7 mg/mL.

9. The injectable composition according to claim 1, wherein the injectable composition contains bivalirudin or a pharmaceutically acceptable salt or a co-crystal thereof; at a concentration range of about 1 mg/mL to about 250 mg/mL.

10. The injectable composition according to claim 1, where in the non-aqueous solvent systems comprises the primary non-aqueous solvent and optionally a secondary non-aqueous co-solvent in a ratio ranging from about 99:1 to about 50:50.

11. The injectable composition according to claim 1, wherein the composition contains not more than 7.0% w/w total impurities (based on total weight of bivalirudin) formed upon storage at real time conditions.

12. The injectable composition according to claim 1, wherein the composition is free of preservatives, anti-oxidants and polymers.

13. A process for the preparation of the non-aqueous, stable and ready-to-use injectable composition of bivalirudin or a pharmaceutically acceptable salt or a co-crystal thereof, comprising the steps of:
a) dissolving a pH adjusting agent in a non-aqueous solvent system consisting of a primary non-aqueous solvent to obtain a first solution;
b) optionally adding polyol to the first solution of step (a) under constant stirring until the polyol dissolves to obtain a second solution;
c) optionally adding secondary non-aqueous solvent to the second solution of step (b) to obtain a third solution;
d) adding bivalirudin to the third solution of step (c) and allowing the bivalirudin to disperse to obtain a solution;
e) filtering the solution of step (d) to obtain a clear solution; and
g) filling the clear solution of step (e) into a container to obtain a composition in a ready-to-use form.

14. A process for the preparation of the non-aqueous, stable and ready-to-use injectable composition of bivalirudin or a pharmaceutically acceptable salt or a co-crystal thereof, comprising the steps of:
a) dissolving polyol and pH adjusting agent in propylene glycol to obtain a first solution;
b) optionally adding a secondary non-aqueous solvent to the first solution of step (a) to obtain a second solution;
c) adding bivalirudin to the second solution of step (b) and allowing the bivalirudin to disperse and solubilize to obtain a solution;
d) filtering the solution of step (c) one or more times to obtain a clear solution; and
e) filling the clear solution of step (d) into a container to obtain a composition in a ready-to-use form.

15. The injectable composition according to claim 1, for use as an anti-coagulant.

16. A method of treating unstable angina in patients undergoing percutaneous transluminal coronary angioplasty (PTCA) comprising administering an effective amount of an injectable composition of claim 1.

17. The injectable composition according to claim 3, wherein the primary non-aqueous solvent system comprises a solvent selected from the group consisting of propylene glycol, glycerol and polyethylene glycol or a mixture thereof.
